(19) United States
(12) Patent Application Publication
(10) Pub. No.: US 2002/0143279 A1
(43) Pub. Date: Oct. 3, 2002

(54) Angle sensor for orthopedic rehabilitation device

(76) Inventors: David A. Porier, Escondido, CA (US); Sumit Purohit, Carlsbad, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 10/116,343

(22) Filed: Apr. 3, 2002

Related U.S. Application Data
(62) Division of application No. 09/558,525, filed on Apr. 26, 2000.

(51) Int. Cl. A61F 5/00

(52) U.S. Cl. 602/16; 602/5

(57) ABSTRACT

An angle sensor for an orthopedic rehabilitation device includes a magnet and Hall effect sensor. The magnet is attached to a first member of the orthopedic rehabilitation device, and the Hall effect sensor is attached to a second member of the orthopedic rehabilitation device. The Hall effect sensor detects the presence of a magnetic flux created by the magnet, and produces an output voltage signal that changes as a function of the magnetic flux detected by the Hall effect sensor. As the first member rotates relative to the second member, the magnet rotates relative to the Hall effect sensor, which causes a change in the magnetic flux detected by the Hall effect sensor. The change in magnetic flux causes a change in the magnitude of the output voltage signal generated by the Hall effect sensor, which is converted into an angular equivalent.

### BACKGROUND OF THE INVENTION

1. Field of the Invention

This invention relates in general to orthopedic rehabilitation devices and, more particularly, to an angle sensor for measuring the angle of a joint.

2. Description of the Related Art

An orthopedic rehabilitation device, such as an orthopedic knee brace, is worn on the joint of a user either to support a healthy joint that is at risk of injury or to stabilize a joint that has been destabilized by an injury or by some other condition. Orthopedic rehabilitation devices generally include rigid structural components linked together by one or more hinges. These hinges enable controlled pivotal movement of the joint during user activity or rehabilitative therapy.

Some orthopedic rehabilitation devices include a position sensor that measures the relative angular position of two components of the device linked together by a hinge. By measuring the angle between certain components of the orthopedic rehabilitation device, the angle of the user's joint can be determined.

Some orthopedic rehabilitation devices include a custom potentiometer as a position sensor. However, such potentiometers exhibit certain drawbacks as position sensors. For example, after repeated cycles, potentiometers have a tendency to wear out, because they are typically designed such that a wiper arm of the potentiometer makes sliding contact along a resistive film when the orthopedic rehabilitation device is in use. In addition, contaminants such as dust or dirt may infiltrate the potentiometer and hinder contact between the wiper arm and the resistive film, thereby causing the potentiometer to provide erroneous or intermittent measurements. Furthermore, the custom potentiometers typically included in orthopedic rehabilitation devices generally require fairly expensive tooling to manufacture.

For these and other reasons, designers have sought to develop a position sensor for orthopedic rehabilitation devices that is accurate, durable, and inexpensive.

### SUMMARY OF THE INVENTION

In accordance with one embodiment, the present invention provides an orthopedic rehabilitation device comprising a first rigid member, a second rigid member, and a hinge coupling the rigid members such that the first rigid member can rotate relative to the second rigid member at a pivot point. The orthopedic rehabilitation device further comprises an angle sensor comprising a magnet and a Hall effect sensor, wherein the magnet is secured to the first rigid member and the Hall effect sensor is secured to the second rigid member.

In accordance with another embodiment, the present invention provides a method for measuring an angle between a first member and a second member of an orthopedic rehabilitation device. The method comprises the steps of providing a magnetic flux which varies according to the angle and detecting the magnetic flux with a Hall effect sensor. The method further comprises the steps of generating an output signal with the Hall effect sensor, wherein the output signal is related to the magnetic flux, and converting the output signal into an angular equivalent.

In accordance with another embodiment, the present invention provides a method for calibrating an angle sensor for an orthopedic rehabilitation device, wherein the orthopedic rehabilitation device comprises a first rigid member rotatably secured relative to a second rigid member and the angle sensor comprises a magnet secured to the first rigid member and a Hall effect sensor secured to the second rigid member. The method comprises the steps of positioning the first rigid member of the orthopedic rehabilitation device in a plurality of predetermined positions relative to the second rigid member of the orthopedic rehabilitation device, detecting an output signal value of a Hall effect sensor at each of the plurality of predetermined positions, and storing the output signal values in an electronic storage device.

In accordance with another embodiment, the present invention provides a hinge mechanism for an orthotic brace, wherein the orthotic brace comprises a first rigid member rotatably secured relative to a second rigid member. The hinge mechanism comprises a pivot and an angle sensor for measuring an angle of a joint, wherein the angle sensor comprises a magnet fixedly secured to the first rigid member of the orthotic brace and a Hall effect sensor fixedly secured to the second rigid member of the orthotic brace.

For purposes of summarizing the invention and the advantages achieved over the prior art, certain objects and advantages of the invention have been described herein above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these embodiments are intended to be within the scope of the invention herein disclosed. These and other embodiments of the present invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

Having thus summarized the general nature of the invention and its essential features and advantages, certain preferred embodiments and modifications thereof will become apparent to those skilled in the art from the detailed description herein having reference to the figures that follow, of which:

FIGS. 1A and 1B are simplified schematic diagrams of part of an orthopedic rehabilitation device having an angle sensor in accordance with one embodiment of the present invention.

FIG. 2A is a perspective view of an orthopedic knee brace having an angle sensor in accordance with one embodiment of the present invention.

FIG. 2B is a detailed cross-sectional view of the orthopedic knee brace illustrated in FIG. 2A along the section line 2B-2B shown in FIG. 2A.

FIG. 3 is a graph showing a typical response curve for the output voltage signal of an angle sensor of FIG. 2B.

FIG. 4 is a block diagram of a circuit board for an orthopedic knee brace and a circuit board for a remote display unit in accordance with one embodiment of the present invention.

FIG. 5 is a circuit diagram of a circuit board for a remote display unit in accordance with one embodiment of the present invention.

FIG. 6 is a circuit diagram of a circuit board for an orthopedic knee brace in accordance with one embodiment of the present invention.

### DETAILED DESCRIPTION OF THE PREFERRED EMBODIMENT

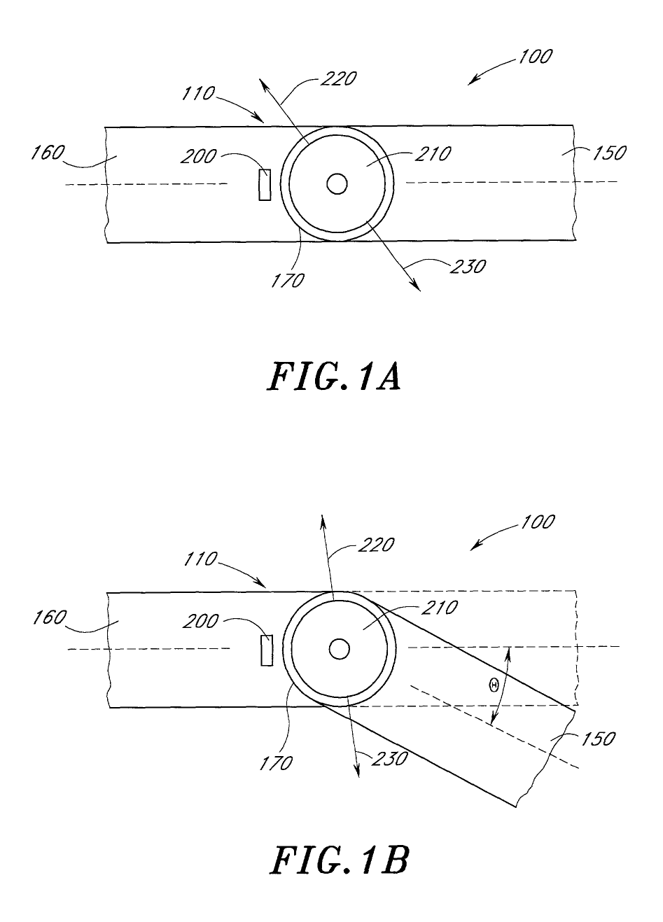

FIGS. 1A and 1B are simplified schematic diagrams of part of an orthopedic rehabilitation device 100 having an angle sensor 110 in accordance with one embodiment of the present invention. In one embodiment, the orthopedic rehabilitation device 100 comprises an orthopedic knee brace. Those of ordinary skill in the art will understand, however, that the orthopedic rehabilitation device 100 may comprise a variety of suitable orthotic devices. The orthopedic rehabilitation device 100 comprises a first bar 150 and a second bar 160. The first bar 150 is pivotally coupled to the second bar 160 by a hinge 170. The angle sensor 110 comprises a Hall effect sensor 200 and a magnet 210 rotatably secured relative to one another.

In one embodiment, the magnet 210 is a circular disk magnet comprising a grade 1 ceramic material and having an outer diameter of about 0.77 inches, an inner diameter of about 0.24 inches, and a thickness of about 0.11 inches. The magnet 210 is preferably attached to the first bar 150 on one side of the orthopedic rehabilitation device 100 and is centered on the pivot point of the first bar 150 and the second bar 160. Furthermore, the magnet 210 is preferably magnetized through its diameter, having a single north pole 220 and a single opposing south pole 230.

The Hall effect sensor 200 detects the presence of magnetic flux and produces an output signal that changes as a function of the magnetic flux detected by the Hall effect sensor 200. Those of ordinary skill in the art will understand that the output signal generated by the Hall effect sensor 200, which is a voltage signal, may be converted to a variety of useful signals, such as, for example, an output current signal or an output digital signal. In one embodiment, the Hall effect sensor 200 is attached to the second bar 160 and is positioned near the edge of the magnet 210. The magnetic flux density detected by the Hall effect sensor 200 varies with the angular orientation of the magnet 210 with respect to the Hall effect sensor 200. Accordingly, the output voltage signal of the Hall effect sensor 200 varies as a function of the relative angular orientation of the magnet 210 with respect to the Hall effect sensor 200.

In a preferred embodiment, the Hall effect sensor 200 is a Melexis MLX 90215 sensor. The internal gain of the Melexis MLX 90215 Hall effect sensor is preferably set to about 50 millivolts per milliTesla (mv/mT), which is approximately the middle of the range of gain values for the sensor. The Quiescent Output Voltage (V_{0Q}) of the Melexis MLX 90215 Hall effect sensor is preferably set to about one half the voltage supplied to the Hall effect sensor. In a preferred embodiment, the V_{0Q} is set to about 2.3 volts, with a supply voltage of about 4.6 volts. Those of ordinary skill in the art will understand, however, that these parameters can be adjusted through routine optimization for any given angle sensor configuration.

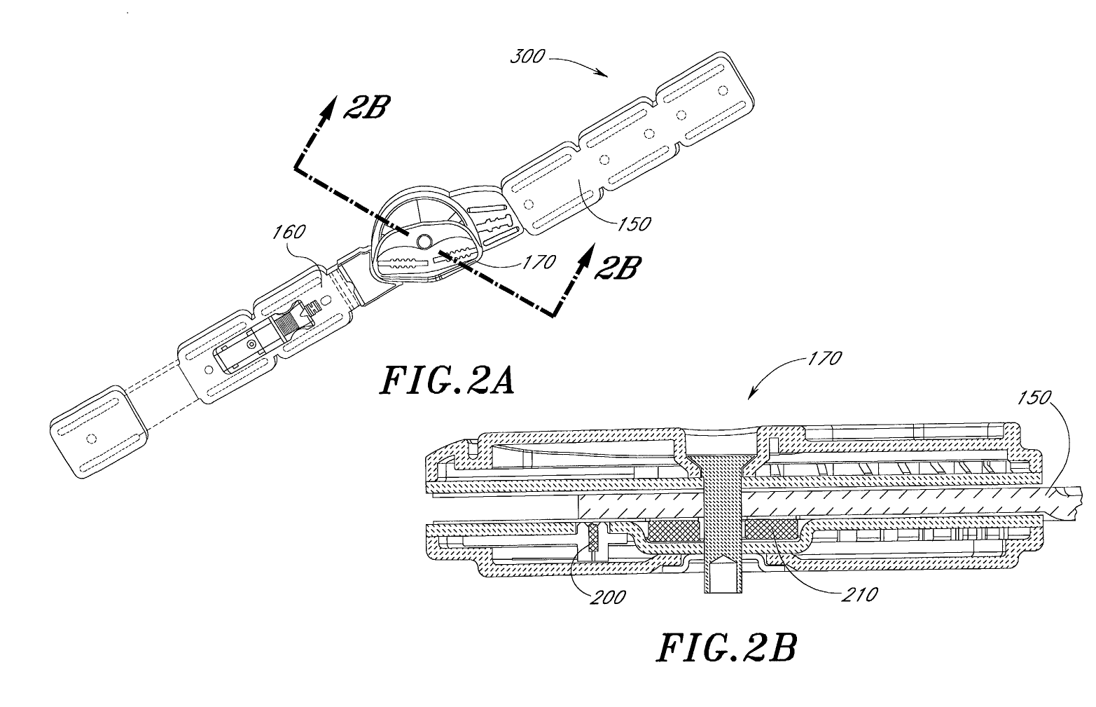

FIG. 2A is a perspective view of an orthopedic knee brace 300 having an angle sensor 110 in accordance with one embodiment of the present invention. FIG. 2B is a detailed cross-sectional view of the orthopedic knee brace 300 illustrated in FIG. 2A along the section line 2B-2B shown in FIG. 2A. The orthopedic knee brace 300 comprises a first bar (“thigh bar”) 150 and a second bar (“calf bar”) 160 pivotally coupled together with a hinge 170. The angle sensor 110 generally comprises a Hall effect sensor 200 and a magnet 210. The Hall effect sensor 200 is fixedly secured relative to the calf bar 160, while the magnet 210 is fixedly secured relative to the thigh bar 150.

When the orthopedic knee brace 300 is in use, the thigh bar 150 is secured to the user's thigh, and the calf bar 160 is secured to the user's calf. The hinge 170 allows the thigh bar 150 to rotate relative to the calf bar 160, thereby enabling controlled flexion and extension of the user's knee. As the user flexes or extends his or her knee, the thigh bar 150 rotates relative to the calf bar 160, which causes the magnet 210 to rotate relative to the Hall effect sensor 200. As the magnet 210 rotates relative to the Hall effect sensor 200, the magnetic flux detected by the Hall effect sensor 200 changes in a predictable fashion. As discussed above, this change in magnetic flux causes a corresponding change in the magnitude of the output signal generated by the Hall effect sensor 200.

Therefore, the output signal generated by the Hall effect sensor 200 correlates to the relative angular position of the magnet 210 and the Hall effect sensor 200. In turn, the relative angular position of the magnet 210 and the Hall effect sensor 200 correlates to the relative angular position of the thigh bar 150 and the calf bar 160, and thus to the flexion angle of the user's knee.

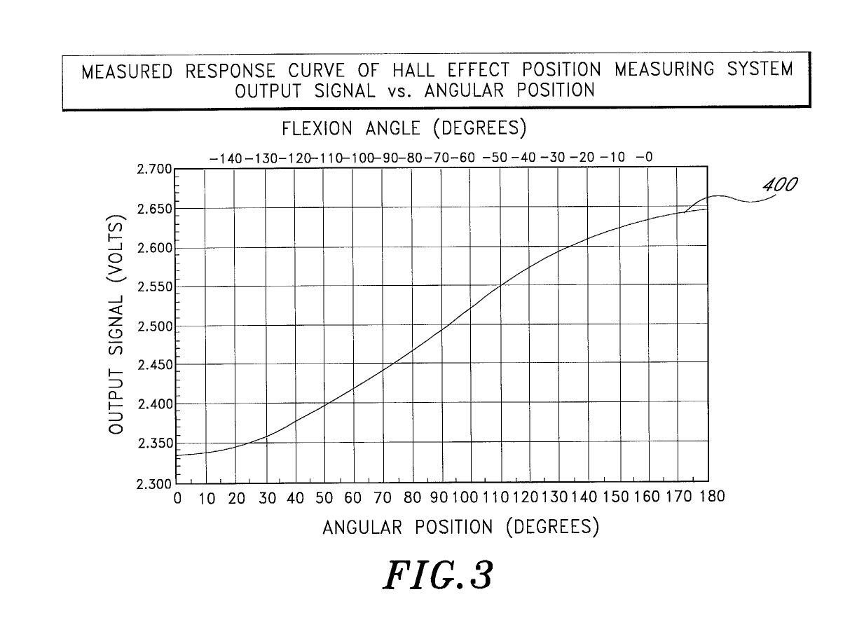

FIG. 3 is a graph showing a typical response curve 400 for the output voltage signal of the Hall effect sensor 200. This graph demonstrates the correlation between the output voltage signal of the Hall effect sensor 200, the relative angular position of the thigh bar 150 and the calf bar 160, and the flexion angle of the user's knee. In the illustrated embodiment, a 0° flexion angle indicates full leg extension and a 140° flexion angle indicates full leg flexion.

As illustrated in FIG. 3, the angle between the thigh bar 150 and the calf bar 160 can be calculated by monitoring the output voltage signal of the Hall effect sensor 200. Because the response curve 400 shown in FIG. 3 is approximately linear near the center portion of the curve 400, the Hall effect sensor 200 provides a parsably distinct, separate output voltage signal value for each angular position of the magnet 210 in this portion of the curve 400. Therefore, the angle sensor 110 of the orthopedic knee brace 300 preferably operates in this substantially linear region of the response curve 400.

To adjust the angle sensor 110 such that it operates in the substantially linear region of the response curve 400, the magnet 210 is preferably placed on the thigh bar 150 such that the Hall effect sensor 200 is positioned approximately halfway between the north pole 220 and the south pole 230 of the magnet 210 when the angle between the thigh bar 150 and the calf bar 160 has reached about ½ of the total range of motion. For example, in the illustrated embodiment—where a 0° flexion angle indicates full leg extension and a 140° flexion angle indicates full leg flexion—the Hall effect sensor 200 is positioned approximately halfway between the north pole 220 and the south pole 230 when the flexion angle (θ) is about 70°, as illustrated in FIG. 1B.

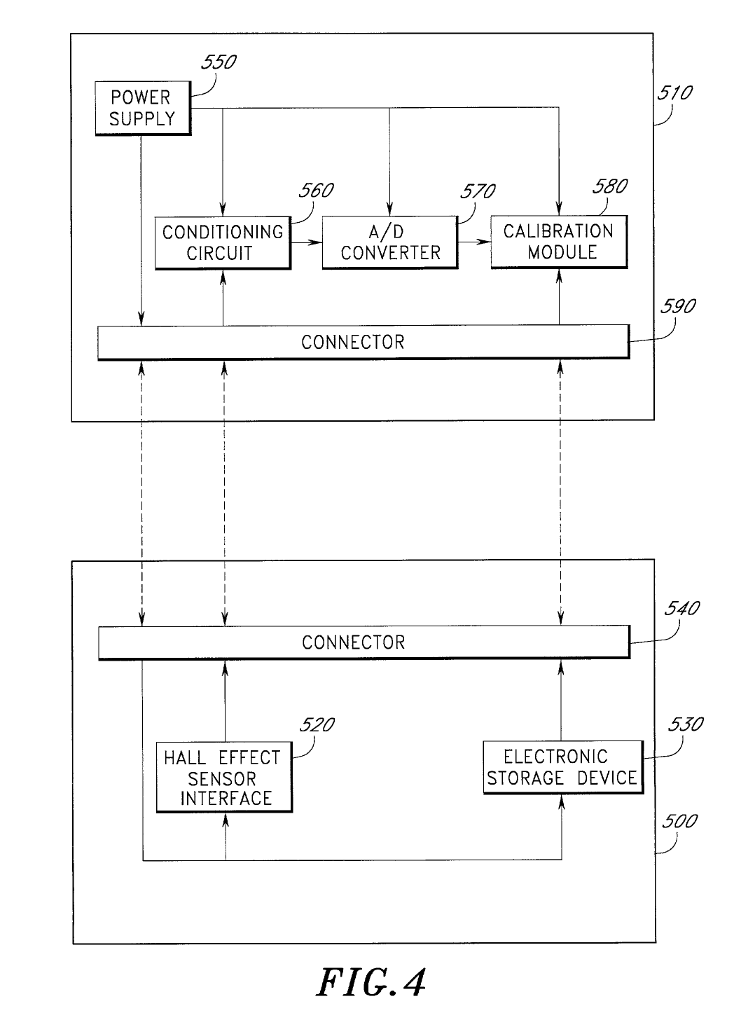

FIG. 4 is a block diagram of a circuit board 500 for an orthopedic knee brace 300 and a circuit board 510 for a remote display unit in accordance with one embodiment of the present invention. In a preferred embodiment, the remote display unit comprises a handheld LCD unit. Those of ordinary skill in the art will understand, however, that the remote display unit may comprise a variety of display units, such as, for example, LCD, LED, gas plasma, CRT or other suitable display units, as desired.

The circuit board 500 for the orthopedic knee brace 300 comprises a Hall effect sensor interface 520 and an electronic storage device 530, each coupled to a connector 540. In a preferred embodiment, the electronic storage device 530 comprises an EEPROM device, such as an STMicroelectronics M24C02 BEPROM device. Those of ordinary skill in the art will understand, however, that the electronic storage device 530 may comprise a variety of suitable devices.

The circuit board 510 for the remote display unit comprises a power supply 550 coupled to a conditioning circuit 560, to an analog-to-digital (A/D) converter 570, to a calibration module 580, and to a connector 590. In a preferred embodiment, the calibration module 580 comprises a microcontroller, such as a Motorola 68HC11. The conditioning circuit 560 is coupled to the A/D converter 570 and to the connector 590. In addition, the A/D converter 570 is coupled to the calibration module 580, which is also coupled to the connector 590.

The connector 540 in the orthopedic knee brace 300 is configured to be coupled to the connector 590 in the remote display unit. In one embodiment, the connectors 540, 590 are configured to be coupled together with a shielded cable (not shown). Those of ordinary skill in the art will understand, however, that the connectors 540, 590 can be coupled together with a variety of cables or wireless communication devices.

The power supply 550 provides a reference voltage signal, referred to as V_{BRACE}, to the conditioning circuit 560, to the A/D converter 570, and to the calibration module 580. When the connectors 540, 590 are coupled together, the power supply 550 also provides the reference voltage signal, V_{BRACE}, to the Hall effect sensor interface 520 and to the electronic storage device 530. Furthermore, when the connectors 540, 590 are coupled together, the Hall effect sensor interface 520 is coupled to the conditioning circuit 560, and the electronic storage device 530 is coupled to the calibration module 580.

In operation, the Hall effect sensor interface 520 provides the output voltage signal of the Hall effect sensor 200 to the conditioning circuit 560. The conditioning circuit 560 is configured to generate an output voltage signal based upon the input voltage signal received from the Hall effect sensor interface 520 and to provide the output voltage signal to the A/D converter 570. The conditioning circuit 560 is advantageously designed such that it generates an output voltage signal within a predetermined range of values, which corresponds to the optimal range of input voltage values for the A/D converter 570. The A/D converter 570 converts the analog input voltage signal received from the conditioning circuit 560 into a digital output signal, which is provided to the calibration module 580.

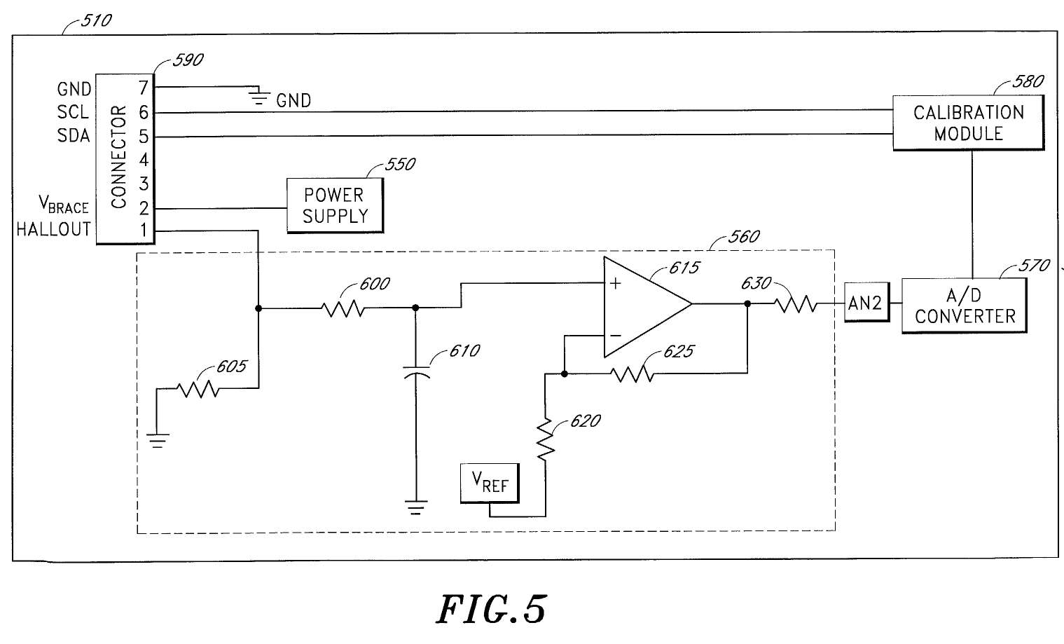

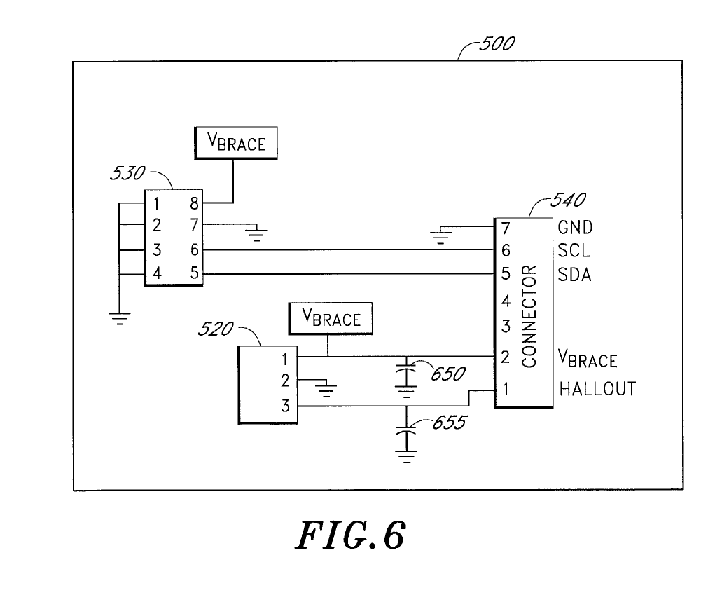

FIG. 5 is a circuit diagram of a circuit board 510 for a remote display unit in accordance with one embodiment of the present invention. As discussed above, the circuit board 510 generally comprises a power supply 550, a conditioning circuit 560, an A/D converter 570, a calibration module 580, and a connector 590. In the illustrated embodiment, pin 2 of the connector 590 is coupled to the power supply 550. Thus, as discussed above, the power supply 550 provides a reference voltage signal, referred to as V_{BRACE}, to the circuit board 500 (FIG. 6) for the orthopedic knee brace 300 when the connectors 540, 590 are coupled together

The conditioning circuit 560 comprises a first resistor 600 coupled to pin 1 of the connector 590, to a second resistor 605, to a capacitor 610, and to a first input of an Operational Amplifier (Op-Amp) 615. The second resistor 605 is also coupled to pin 1 of the connector 590 and to ground. The capacitor 610 is also coupled to the first input of the Op-Amp 615 and to ground. A second input of the Op-Amp 615 is coupled to a third resistor 620 and to a fourth resistor 625. The third resistor 620 is also coupled to a reference voltage signal, referred to as V_{REF}. In a preferred embodiment, the value of V_{REF} is about ½ the value of V_{BRACE}. An output of the Op-Amp 615 is coupled to the fourth resistor 625 and to a fifth resistor 630. The fifth resistor 630 is also coupled to the A/D converter 570.

In operation, pin 1 of the connector 590 provides an input voltage signal from the Hall effect sensor interface 520, referred to as the HALLOUT signal, to the conditioning circuit 560 when the connectors 540, 590 are coupled together. The second resistor 605 acts as a pull-down resistor to drive the output signal of the conditioning circuit 560, referred to as the AN2 signal, to a known state if the connectors 540, 590 become disconnected. The first resistor 600 and the capacitor 610 act as a low-pass filter to remove unwanted high frequencies from the input voltage signal. The third resistor 620 and the fourth resistor 625 are configured to control the gain of the Op-Amp 615. As described above, the gain of the Op-Amp 615 is preferably selected such that the conditioning circuit 560 generates an output voltage signal within the optimal range of input voltage values for the A/D converter 570. The fifth resistor 630 controls the output impedance of the Op-Amp 615.

In operation, the A/D converter 570 receives an analog input voltage signal, referred to as the AN2 signal, from the conditioning circuit 560. As described above, the A/D converter 570 converts the analog input voltage signal received from the conditioning circuit 560 into a digital output signal, which is provided to the calibration module 580.

Pins 5 and 6 of the connector are coupled to the calibration module 580. When the connectors 540, 590 are coupled together, pin 5 of the connector 540 receives a first serial communication signal, referred to as the SDA signal, from the electronic storage device 530. Similarly, pin 6 of the connector 540 receives a second serial communication signal referred to as the SCL signal, from the electronic storage device 530 when the connectors 540, 590 are coupled together. In operation, the SCL signal and the SDA signal are provided to the calibration module 580.

In general, the output voltage signal of the Hall effect sensor 200 in each angle sensor 110 configured in accordance with the present invention generally adheres to the response curve 400 shown in FIG. 3. Some slight variations from this response curve 400 can occur, however, from one angle sensor 110 to another. These variations can potentially create a slight differential between the actual output voltage signal of the Hall effect sensor 200 and the expected output voltage signal for a given angle, thereby reducing the precision and accuracy of the angle sensor 110.

Therefore, in a preferred embodiment, the precision and accuracy of the angle sensor 110 are advantageously optimized by performing a calibration process once the angle sensor 110 is assembled and installed in the orthopedic knee brace 300. During this calibration process, a number of predetermined calibration points are selected, and the flexion angles at the predetermined calibration points are independently measured. For example, in one embodiment, the calibration points are selected at 10° increments from full extension (0°) to full flexion (140°). The orthopedic knee brace 300 is then moved through its entire range of motion, and the actual values of the output voltage signal of the Hall effect sensor 200 at the predetermined calibration points are measured and stored in the electronic storage device 530 (FIG. 6).

In operation, the calibration module 580 retrieves the values stored in the electronic storage device 530 and performs a linear interpolation process to create a complete, individualized position data table for that particular orthopedic knee brace 300. Table 1 shows an excerpt of an exemplary position data table for the calibration points at 20°, 30°, and 40°.

TABLE 1

| | | Interpolated | Difference Between |
|---|---|---|---|
| Angle | Measured Output | Output | Measured Output and |
| (Degrees) | (Volts) | (Volts) | Interpolated Output (Volts) |
| 20 | 3.926 | 3.926 | 0.000 |
| 21 | | 3.899 | |
| 22 | | 3.871 | |
| 23 | | 3.843 | |
| 24 | | 3.815 | |
| 25 | 3.787 | 3.787 | 0.000 |
| 26 | | 3.760 | |
| 27 | | 3.732 | |
| 28 | | 3.704 | |
| 29 | | 3.676 | |
| 30 | 3.648 | 3.648 | 0.000 |
| 31 | | 3.618 | |
| 32 | | 3.587 | |
| 33 | | 3.557 | |
| 34 | | 3.526 | |
| 35 | 3.509 | 3.495 | 0.014 |
| 36 | | 3.465 | |
| 37 | | 3.434 | |
| 38 | | 3.404 | |
| 39 | | 3.373 | |
| 40 | 3.343 | 3.343 | 0.000 |

In Table 1, the “Measured Output” column represents the actual output voltage signal of the Hall effect sensor 200 at 20°, 25°, 30°, 35°, and 40°. The values in the “Interpolated Output” column represent the result of the linear interpolation process used to generate the individualized position data table. This linear interpolation process is performed between every 10° interval, with the maximum error thereby occurring midway between interpolation points.

In operation, the calibration module 580 converts the input signal received from the A/D converter 570 into an angular equivalent based upon the response curve 400 illustrated in FIG. 3. The calibration module 580 preferably refers to the data recorded in the position data table when making this conversion. In one embodiment, the position data table created by the calibration module 580 is a lookup table having an entry for every unit of measure (e.g., every degree) on an actual response curve. In another embodiment, the position data table created by the calibration module 580 contains a series of offset and range correction factors to normalize an actual response curve and fit it to a theoretical or mathematical representation of a nominal response curve (e.g., a cosine wave or a sine wave). In yet another embodiment, the position data table created by the calibration module 580 contains values that correspond to a mathematical response curve generated by reading discrete points on an actual response curve (e.g., least squares or polynomial curve fit).

FIG. 6 is a circuit diagram of a circuit board 500 for an orthopedic knee brace 300 in accordance with one embodiment of the present invention. As described above, the circuit board 500 comprises a Hall effect sensor interface 520, an electronic storage device 530, and a connector 540. In the illustrated embodiment, the electronic storage device 530 is an STMicroelectronics M24C02 EEPROM device.

Pins 1 through 4 and pin 7 of the electronic storage device 530 are coupled to ground. Pin 8 of the electronic storage device 530 is coupled to the V_{BRACE} reference voltage signal. As described above, the power supply 550 in the remote display unit provides the V_{BRACE} reference voltage signal to the electronic storage device 530 when the connectors 540, 590 are coupled together. Pin 5 of the electronic storage device 530, which provides a first serial communication signal (referred to as the SDA signal), is coupled to pin 5 of the connector 540. Similarly, pin 6 of the electronic storage device 530, which provides a second serial communication signal (referred to as the SCL signal), is coupled to pin 6 of the connector 540.

Pin 1 of the Hall effect sensor interface 520 is coupled to the V_{BRACE} reference voltage signal and to a first capacitor 650. As described above, the power supply 550 in the remote display unit provides the V_{BRACE} reference voltage signal to the Hall effect sensor interface 520 when the connectors 540, 590 are coupled together. The first capacitor 650 is also coupled to ground. Pin 2 of the Hall effect sensor interface 520 is coupled to ground. Pin 3 of the Hall effect sensor interface 520 is coupled to a second capacitor 655 and to pin 1 of the connector 540. The second capacitor 655 is also coupled to ground.

In operation, pin 3 of the Hall effect sensor interface 520 provides the output voltage signal of the Hall effect sensor 200, referred to as the HALLOUT signal, to pin 1 of the connector 540. The second capacitor 655 acts as a filter to remove unwanted frequencies from the output voltage signal.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above, but should be determined only by a fair reading of the claims that follow.

What is claimed is:

1. An orthopedic rehabilitation device comprising:
a first rigid member;
a second rigid member;
a hinge coupling said first rigid member to said second rigid member such that said first rigid member can rotate relative to said second rigid member at a pivot point; and
an angle sensor comprising a magnet and a Hall effect sensor, wherein said magnet is secured to said first rigid member and said Hall effect sensor is secured to said second rigid member.

2. The orthopedic rehabilitation device of claim 1, wherein said orthopedic rehabilitation device comprises an orthopedic knee brace.

3. The orthopedic rehabilitation device of claim 1, wherein said magnet comprises a circular disk magnet attached to said first rigid member and centered on said pivot point.

4. The orthopedic rehabilitation device of claim 1, wherein said Hall effect sensor is secured to said second rigid member at a position near said magnet.

5. The orthopedic rehabilitation device of claim 1, further comprising an electronic storage device.

6. A method for measuring an angle between a first member and a second member of an orthopedic rehabilitation device, comprising the steps of:
providing a magnetic flux which varies according to said angle;
detecting said magnetic flux with a Hall effect sensor;
generating an output signal with said Hall effect sensor, wherein said output signal is related to said magnetic flux; and
converting said output signal into an angular equivalent.

7. The method of claim 6, further comprising the steps of:
rotating said first member relative to said second member to create a change in said magnetic flux; and
detecting said change in said magnetic flux with said Hall effect sensor.

8. A method for calibrating an angle sensor for an orthopedic rehabilitation device, wherein said orthopedic rehabilitation device comprises a first rigid member rotatably secured relative to a second rigid member and said angle sensor comprises a magnet secured to said first rigid member and a Hall effect sensor secured to said second rigid member, said method comprising the steps of:
positioning said first rigid member of said orthopedic rehabilitation device in a plurality of predetermined positions relative to said second rigid member of said orthopedic rehabilitation device;
detecting an output signal value of said Hall effect sensor at each of said plurality of predetermined positions; and
storing said output signal values in an electronic storage device.

9. The method of claim 8, further comprising the step of:
interpolating said output signal values to create a position data table.

10. A hinge mechanism for an orthotic brace, wherein said orthotic brace comprises a first rigid member rotatably secured relative to a second rigid member, said hinge mechanism comprising:
a pivot; and
an angle sensor for measuring an angle of a joint, said angle sensor comprising:
a magnet fixedly secured to said first rigid member of said orthotic brace, and
a Hall effect sensor fixedly secured to said second rigid member of said orthotic brace.
